(19) United States
(12) Patent Application Publication
(10) Pub. No.: US 2026/0060715 A1
(43) Pub. Date: Mar. 5, 2026

(54) CANNULA WITH POLYGONAL CANNULA TUBE AND RETROREFLECTING CANNULA TIP

(71) Applicant: B. Braun Melsungen AG, Melsungen (DE)

(72) Inventors: Andre Weiss, Baunatal (DE); Niclas Berger, Kassel (DE); Dejana Vukovic, Kassel (DE); Tobias Schroeder, Hasbergen (DE)

(21) Appl. No.: 19/311,352

(22) Filed: Aug. 27, 2025

(30) Foreign Application Priority Data
Aug. 28, 2024 (DE) 10 2024 124 599.9

(51) Int. Cl. A61B 17/34

(52) U.S. Cl. CPC A61B 17/3421; A61B 2017/3413; A61B 2017/3454

(57) ABSTRACT

A cannula has an elongate cannula tube and a cannula tip. The cannula tube can have a polygonal hollow profile. The cannula tip can have a retroreflecting geometry.

### CROSS-REFERENCE TO RELATED APPLICATIONS

This application claims priority under 35 U.S. C. § 119 to German Application No. 10 2024 124 599.9, filed on Aug. 28, 2024, the content of which is incorporated by reference herein in its entirety.

### FIELD

The present disclosure relates to a cannula having an elongate cannula tube and a distal cannula tip.

### BACKGROUND

Cannulas are also referred to as hollow needles and are used for a very wide variety of medical applications, primarily to puncture body tissue and subsequently inject or aspirate liquids. Cannulas may also be used to fit catheters or other invasive components, in which case the cannula tube serves as a kind of delivery channel for the invasive component in question.

In many medical applications, accurate positioning of the cannula in the body tissue is of particular importance. Cannulas are therefore often positioned by using ultrasound imaging methods, the position of the cannula in the ultrasound image being monitored. This presupposes sufficient visibility of the cannula in the ultrasound image. In other words, the cannula must have a sufficient ultrasound visibility.

Conventional cannulas have a hollow cylindrical profile with an annular cross section and are made of metal, so that generally they already have a sufficient ultrasound visibility.

Cannulas which are intended to achieve an improved ultrasound visibility by modification of the surface condition of the cannula tube are known from the prior art.

### SUMMARY

It is an object of the present disclosure to provide a cannula of the type mentioned, which offers advantages over the prior art. Especially, an improved visual distinguishability between the cannula tube and the cannula tip in the ultrasound image is intended to be made possible.

This object is achieved in that the cannula tube has a polygonal hollow profile. Alternatively or in addition, the cannula tip has a retroreflecting geometry. By the solution according to the present disclosure, the cannula tube and the cannula tip have different reflection properties for ultrasound signals used in position monitoring. The polygonal hollow profile of the cannula tube generates a relatively weak ultrasound echo. The retroreflecting geometry of the cannula tip generates a relatively strong ultrasound echo. In this way, the cannula tip and the cannula tube are visible with different clarity in the ultrasound image, which allows simple and reliable visual discrimination between the cannula tip and the cannula tube in the ultrasound image, and as a result improved position monitoring. In one preferred embodiment, the polygonal hollow profile is a triangular hollow profile having three outer longitudinal edges. In further embodiments, the polygonal hollow profile has more than three outer longitudinal edges, for example four, five or more than five outer longitudinal edges. The retroreflecting geometry of the cannula tip varies in different embodiments. By its retroreflecting geometry, the cannula tip acts as a retroreflector for the ultrasound signals of the ultrasound imaging method. Retroreflectors are known per se, and are also referred to as reflex reflectors. A retroreflector may be understood as a body or a portion of a body, which reflects incident waves, in particular sound waves, substantially independently of the direction of incidence in relation to an alignment of the body or body portion, predominantly in the direction from which the waves have been emitted. This effect is also referred to as retroreflection or reflex reflection. Various embodiments of retroreflectors are known, for example triple mirrors, triple prisms, lens-like embodiments or the like.

In one embodiment of the present disclosure, the polygonal hollow profile is a triangular hollow profile which has three elongate outer longitudinal edges and three outer faces, the outer faces each extending between two of the three outer longitudinal edges. The configuration as a triangular hollow profile allows a maximum reduction of the ultrasound echo of the cannula tube. In this way, the contrast between the cannula tube and the cannula tip in the ultrasound image is particularly strong. The cannula tip may therefore be identified particularly reliably. By the configuration as a triangular hollow profile, almost no ultrasound echo is sent back to the ultrasound source of the ultrasound imaging method, since generally none of the three outer faces is aligned perpendicularly with respect to a sound plane of the ultrasound head of the ultrasound imaging method. Preferably, the three outer faces are each planar. Preferably, the three outer longitudinal edges are each rectilinearly elongate.

In a further embodiment of the present disclosure, the triangular hollow profile has three elongate inner longitudinal edges and three inner faces, the three inner faces each extending between two of the three inner longitudinal edges. In this embodiment of the present disclosure, consequently, not only an outer contour but also the inner contour of the cannula tube is triangular, or has three sides. It is, however, also conceivable and possible that the triangular hollow profile has a circular or otherwise configured cross-sectional opening.

In a further embodiment of the present disclosure, the triangular hollow profile is isosceles, so that the outer faces meet the outer longitudinal edges and/or the inner faces meet the inner longitudinal edges respectively at an angle of 60°. The isosceles configuration of the triangular hollow profile leads to a small ultrasound echo, which is as invariant as possible in relation to different alignments of the cannula tube. Furthermore, the manufacture of the cannula is simplified. In this way costs may be saved.

In a further embodiment of the present disclosure, the polygonal hollow profile is rounded on its outer longitudinal edges respectively with an outer radius. This embodiment of the present disclosure is based on the idea that non-rounded outer longitudinal edges constitute a potential injury risk if the cannula is rotated in the body tissue. By the rounding of the outer longitudinal edges respectively with an outer radius, such injuries are prevented. The ultrasound echo of the polygonal hollow profile is not increased by the outer radii, or is at most increased to an almost negligible extent.

In a further embodiment of the present disclosure, the retroreflecting geometry of the cannula tip is formed by material removal, in particular a bevel, of the polygonal hollow profile. In order to form the retroreflecting geometry, a distal end of the cannula tube may be obliquely removed, in particular bevelled. In this case, the oblique material removal and/or bevel of the polygonal hollow profile forms the retroreflecting geometry of the cannula tip.

In a further embodiment of the present disclosure, the retroreflecting geometry has at least a first face, a second face and a third face, the first face being oriented perpendicularly with respect to the second face and perpendicularly with respect to the third face, and the second face and the third face being turned towards one another. By the first face, second face and third face and their orientation with respect to one another, the ultrasound echo of the cannula tip may be further maximised. Preferably, the second face and the third face lie directly opposite one another. In other words: the second face and the third face preferably lie at the same height in relation to the first face. Preferably, the first face and the second face and the third face are each planar.

In a further embodiment of the present disclosure, the first face is formed by a surface portion of an inner face of the polygonal hollow profile, and the second face and the third face are respectively formed by a surface portion of a wall face, the wall faces each extending between an inner face and an outer face of the polygonal hollow profile. The second face and the third face are in this embodiment of the present disclosure preferably exposed by material removal and/or a bevel of the distal cannula tube.

### BRIEF DESCRIPTION OF THE DRAWINGS

Further advantages and features of the present disclosure may be found in the following description of a preferred exemplary embodiment of the present disclosure, which is represented with the aid of the drawings.

FIG. 1 shows a schematically simplified, proximally truncated side view of an embodiment of a cannula according to the present disclosure with a cannula tube and a distal cannula tip;

FIG. 2 shows the cannula tube of the cannula of FIG. 1 in a cross section along a section line II-II according to FIG. 1;

FIG. 3 shows the cannula tip of the cannula of FIG. 1 in a cross section along a section line III-III according to FIG. 1;

FIG. 4 shows an exemplary working situation, schematically represented in a simplified way, of the cannula of FIG. 1 to illustrate the reduced ultrasound visibility of the cannula tube;

FIG. 5 shows a representation of a cannula tube known from the prior art with an annular hollow profile, which has a comparatively strong ultrasound echo;

FIG. 6 shows a schematic representation of a further exemplary working situation of the cannula of FIG. 1 to illustrate the retroreflecting properties of the cannula tip, the cannula assuming a first orientation in relation to an imaging ultrasound source;

FIG. 7 shows a schematic representation of a further exemplary working situation corresponding to FIG. 6, the cannula assuming a second orientation in relation to the ultrasound source; and

FIG. 8 shows a simplified and truncated perspective view of the cannula of FIG. 1 in the region of the distal cannula tip.

### DETAILED DESCRIPTION

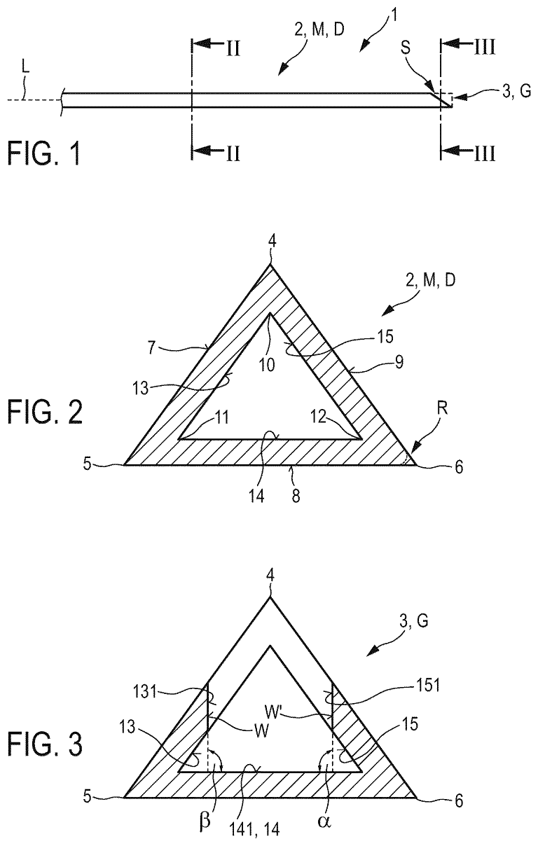

According to FIG. 1, a cannula 1 has an elongate cannula tube 2 and a distal cannula tip 3.

The cannula 1 may also be referred to as a hollow needle and serves to puncture body tissue for the purpose of subsequent injection and/or aspiration of liquid. Alternatively or in addition, the cannula 1 may be used to introduce invasive components, for example guide wires or catheters, into the body of a patient.

The cannula tube 2 is rectilinearly elongate along a longitudinal axis L. In FIG. 1, the cannula tube 2 is represented proximally truncated. The cannula tip 3 is arranged at a distal end of the cannula tube 2. In other words: the cannula tip 3 forms the distal end of the cannula tube 2.

The cannula tube 2 has a polygonal hollow profile M, the cross section of which is shown in detail in FIG. 2. The cannula tip 3 has a retroreflecting geometry G, the cross section of which is shown in detail in FIG. 3.

The polygonal hollow profile M has a weak ultrasound echo in comparison to the retroreflecting geometry G. Conversely, the retroreflecting geometry G has a strong ultrasound echo in comparison to the polygonal hollow profile M. If the cannula 1 is positioned in the body tissue by using an ultrasound imaging method, the medical staff performing the intervention can discriminate easily and reliably between the cannula tube 2 and the cannula tip 3 in the resulting ultrasound image. This allows improved positioning of the cannula 1.

In the embodiment shown, the polygonal hollow profile M is a triangular hollow profile D. The triangular hollow profile D has three elongate outer longitudinal edges 4, 5, 6 and three outer faces 7, 8, 9. The outer longitudinal edges 4, 5, 6 may also be referred to as the first outer longitudinal edge 4, second outer longitudinal edge 5 and third outer longitudinal edge 6. The outer faces 7, 8, 9 may also be referred to as the first outer face 7, second outer face 8 and third outer face 9.

The outer faces 7, 8, 9 are each planar. In one embodiment, which is not shown in the figures, the outer faces are each concavely curved. Alternatively, the outer faces may be convexly curved.

The first outer face 7 extends between the first outer longitudinal edge 4 and the second outer longitudinal edge 5. The second outer face 8 extends between the second outer longitudinal edge 5 and the third outer longitudinal edge 6. The third outer face 9 extends between the third outer longitudinal edge 6 and the first outer longitudinal edge 4.

In the embodiment shown, the outer longitudinal edges 4, 5, 6 each extend along the longitudinal axis L over the entire length of the cannula tube 2.

In the embodiment shown, the triangular hollow profile D has three elongate inner longitudinal edges 10, 11, 12 and three inner faces 13, 14, 15. In other words: both an outer contour and an inner contour of the cannula tube 2 are configured triangularly, or with three sides.

In one embodiment, which is not shown in the figures, unlike the outer contour, the inner contour is round, particular circular or oval. In a further embodiment, the inner contour has a polygonal configuration with more than three inner longitudinal edges, for example four, five, six or more than six inner longitudinal edges.

The inner longitudinal edges 10, 11, 12 may also be referred to as the first inner longitudinal edge 10, second inner longitudinal edge 11 and third inner longitudinal edge 12. The inner faces 13, 14, 15 may also be referred to as the first inner face 13, second inner face 14 and third inner face 15.

The first inner face 13 extends between the first inner longitudinal edge 10 and the second inner longitudinal edge 11. The second inner face 14 extends between the second inner longitudinal edge 11 and the third inner longitudinal edge 12. The third inner face 15 extends between the third inner longitudinal edge 12 and the first inner longitudinal edge 10. The inner faces 13, 14, 15 are each planar. The inner faces 13, 14, 15 are in the present case elongate over the entire length of the cannula tube 2 along the longitudinal axis L.

The first outer face 7 and the first inner face 13 are parallel to one another. The same applies mutatis mutandis for the second outer face 8 and the second inner face 14, as well as for the third outer face 9 and the third inner face 15.

In the embodiment shown, the triangular hollow profile D is isosceles. Accordingly, the outer faces 7, 8, 9 are oriented at an angle of 60° with respect to the outer longitudinal edges 4, 5, 6 and the inner faces 13, 14, 15 are oriented at an angle of 60° with respect to the inner longitudinal edges 10, 11, 12.

In the embodiment shown, the triangular hollow profile D is rounded on its outer longitudinal edges 4, 5, 6 respectively with an outer radius R. The outer radius R is graphically indicated in FIG. 2 only in relation to the third outer longitudinal edge 6.

The retroreflecting geometry G is formed in the embodiment shown by material removal S of the polygonal hollow profile M. The material removal S is in the present case a bevel. The cannula tip 3 is consequently formed by oblique grinding of the distal end of the cannula tube 2.

In the embodiment shown, the retroreflecting geometry G has at least a first face 141, a second face 131 and a third face 151. The first face 141 is oriented perpendicularly with respect to the second face 131 and perpendicularly with respect to the third face 151. The angles α, β marked in FIG. 3 accordingly both correspond to 90°. The second face 131 and the third face 151 are turned towards one another. The second face 131 and the third face 151 in this case lie directly opposite one another. In relation to the first face 141, the second face 131 and the third face 151 lie at the same height.

In the embodiment shown, the first face 141, the second face 131 and the third face 151 are each planar.

In the embodiment shown, the first face 141 is formed by a surface portion of the third inner face 14. The second face 131 and the third face 151 are respectively formed by a surface portion of a wall face W, W′, which extends between inner faces and outer faces of the polygonal hollow profile M. The second face 131 in this case extends between the first inner face 13 and the first outer face 7. The third face 151 extends between the third inner face 15 and the third outer face 9. The wall faces W, W′, and therefore the second face 131 and the third face 151, are exposed by the said material removal S.

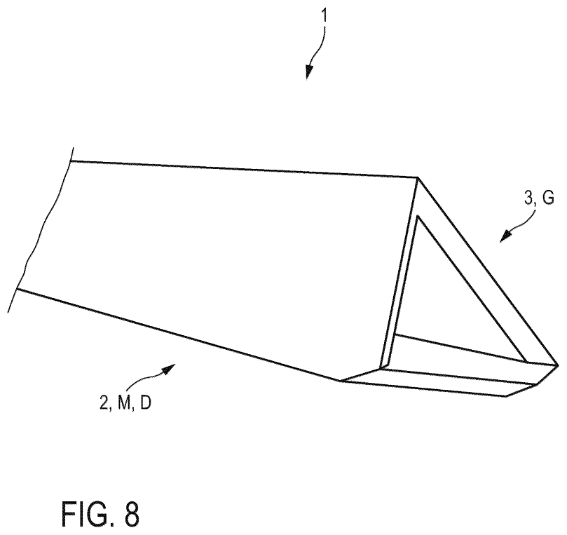

The configuration of the retroreflecting geometry G as shown in FIGS. 3, 6, 7 and 8 is to be regarded as schematic. In embodiments, which are not shown in the figures, the retroreflecting geometry has a different configuration, in which case more or fewer faces and different orientations of the faces may in particularly be provided. In other words: the cannula tip 3 may be configured in a manner other than as a retroreflector. The configuration shown in the figures is to be understood as purely exemplary.

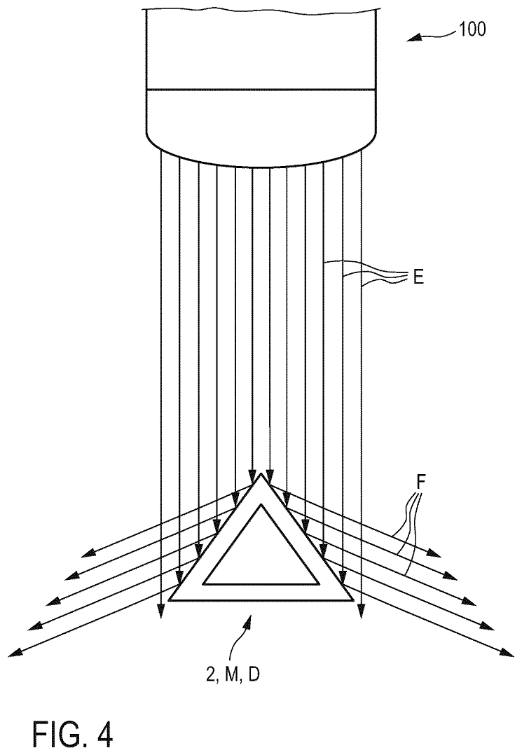

FIG. 4 shows the cannula tube 2 in cross section together with an ultrasound head 100 of an ultrasound imaging system in an exemplary working situation. The ultrasound head 100 transmits ultrasound signals, which are reflected at the cannula tube 2. The transmitted ultrasound signals are provided with the reference sign E in FIG. 4. The reflected ultrasound signals are provided with the reference sign F. By the configuration of the cannula tube 2 as a polygonal hollow profile M, especially as a triangular hollow profile D, the transmitted ultrasound signals E in the working situation shown are reflected by the ultrasound head 100 in such a way as to provide guidance. Because of its specific configuration, the cannula tube 2 has no ultrasound echo that can be determined by the ultrasound head 100. The cannula tube 2 consequently has no ultrasound visibility, or only a very low ultrasound visibility.

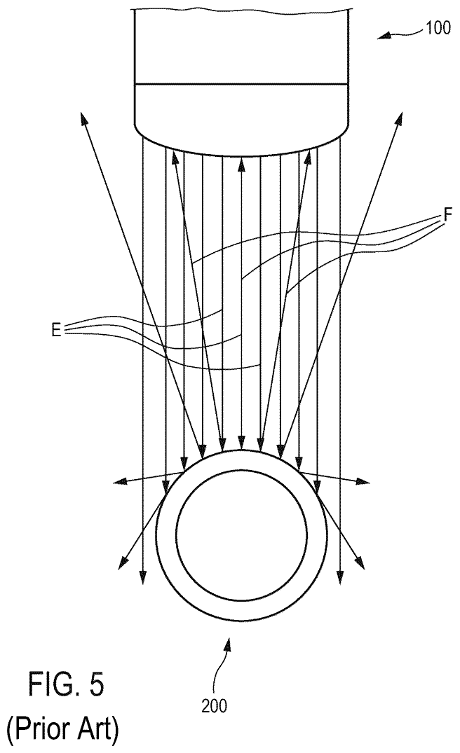

As opposed to this, FIG. 5 shows a cannula tube 200 known from the prior art with an annular cross section. Because of the annular configuration of the cannula tube 200, the transmitted ultrasound signals E are at least partially reflected back in the direction of the ultrasound head 100. The reflected ultrasound signals F can be received by the ultrasound head 100. The cannula tube 200 known from the prior art therefore has a stronger ultrasound echo, and therefore a clearer ultrasound visibility, than the cannula tube 2 of FIG. 1 to 4.

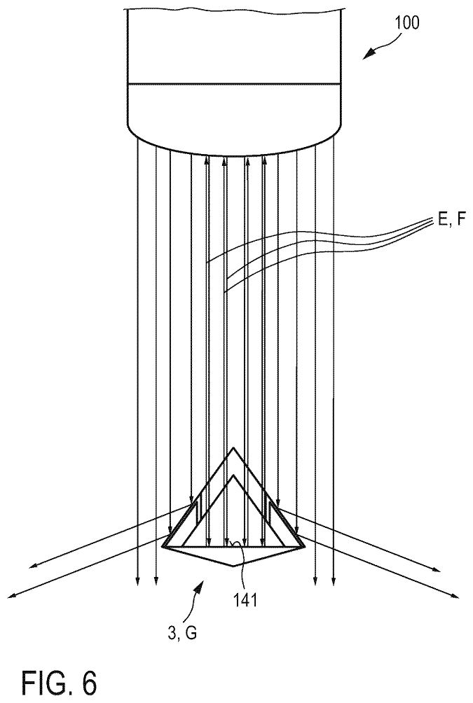

FIG. 6 shows the cannula tip 3 in cross section together with the ultrasound head 100. The effect of the retroreflecting geometry G of the cannula tip 3 is that the transmitted ultrasound signals E are reflected back as completely as possible in the direction of the ultrasound head 100. The reflected ultrasound signals F cause a comparatively strong ultrasound echo and therefore a clear ultrasound visibility of the cannula tip 3. In the working situation shown in FIG. 6, the first face 141 of the retroreflecting geometry G is directly below and aligned approximately parallel to the ultrasound head 100. The reflected ultrasound signals F are primarily reflected back in the direction of the ultrasound head 100 via the first face 141.

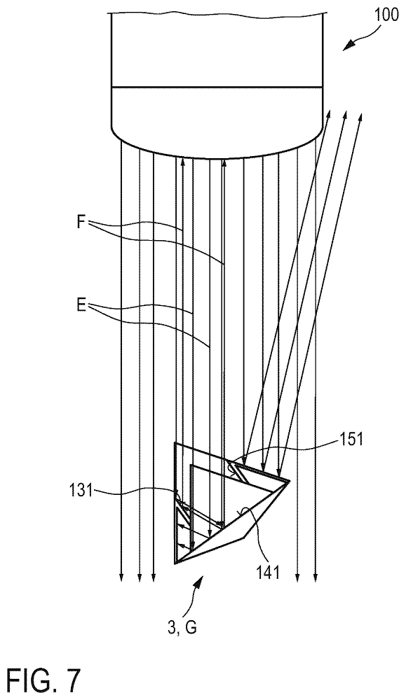

In FIG. 7, the cannula 1 is rotated anticlockwise about its longitudinal axis L relative to the position shown in FIG. 6. This gives a different orientation of the retroreflecting geometry G and its faces 141, 131, 151 in relation to the ultrasound head 100. Substantially independently of this different rotational position, there is nevertheless a strong ultrasound echo of the cannula tip 3. This is due to the configuration as a retroreflector. In the working situation shown, ultrasound signals E are reflected by the first face 141 in the direction of the second face 131 and from there back to the ultrasound head 100.

1. A cannula comprising:
an cannula tube that is elongated; and
a cannula tip,
the cannula tube having a polygonal hollow profile.

2. The cannula according to claim 1, wherein the polygonal hollow profile is a triangular hollow profile which has three elongate outer longitudinal edges and three outer faces, each outer face extending between two of the three elongate outer longitudinal edges.

3. The cannula according to claim 2, wherein the triangular hollow profile has three elongate inner longitudinal edges and three inner faces, each inner face extending between two of the three elongate inner longitudinal edges.

4. The cannula according to claim 3, wherein the triangular hollow profile is isosceles, so that:
the three outer faces meet the three elongate outer longitudinal edges at an angle of 60°; and/or
the three inner faces meet the three elongate inner longitudinal edges respectively at an angle of 60°.

5. The cannula according to claim 1, wherein the polygonal hollow profile comprises outer longitudinal edges that are rounded respectively with an outer radius.

6. The cannula according to claim 1, wherein the cannula tip has a retroreflecting geometry.

7. The cannula according to claim 6, wherein the retroreflecting geometry of the cannula tip is formed by material removal of the polygonal hollow profile.

8. The cannula according to claim 6, wherein the retroreflecting geometry of the cannula tip is formed by a bevel.

9. The cannula according to claim 6, wherein the retroreflecting geometry has at least a first face, a second face and a third face, the first face being oriented perpendicularly with respect to the second face and perpendicularly with respect to the third face, and the second face and the third face being turned towards one another.

10. The cannula according to claim 9, wherein the first face is formed by a surface portion of an inner face of the polygonal hollow profile, and wherein the second face and the third face are respectively formed by a surface portion of a wall face, which extends between an inner face and an outer face.

11. A cannula comprising:
a cannula tube that is elongated; and
a cannula tip,
the cannula tip having a retroreflecting geometry.

12. The cannula according to claim 11, wherein:
the cannula tube has a polygonal hollow profile; and
the retroreflecting geometry of the cannula tip is formed by material removal of the polygonal hollow profile.

13. The cannula according to claim 12, wherein the retroreflecting geometry has at least a first face, a second face and a third face, the first face being oriented perpendicularly with respect to the second face and perpendicularly with respect to the third face, and the second face and the third face being turned towards one another.

14. The cannula according to claim 13, wherein the first face is formed by a surface portion of an inner face of the polygonal hollow profile, and wherein the second face and the third face are respectively formed by a surface portion of a wall face, which extends between an inner face and an outer face.

15. The cannula according to claim 11, wherein the retroreflecting geometry of the cannula tip is formed by a bevel.
